(19) United States
(12) Patent Application Publication
(10) Pub. No.: US 2016/0157882 A1
(43) Pub. Date: Jun. 9, 2016

(54) HIGH-INTENSITY FOCUSED ULTRASOUND (HIFU) PROBES WITH AUTOMATED CONTROL

(71) Applicant: KONINKLIJKE PHILIPS N.V., Eindhoven (NL)

(72) Inventors: JUHA MIKAEL OILA, EINDHOVEN (NL); ERKKI TAPANI VAHALA, Eindhoven (NL)

(73) Assignee: KONINKLIJKE PHILIPS N.V., EINDHOVEN (NL)

(21) Appl. No.: 14/905,043

(22) PCT Filed: Jul. 4, 2014
(86) PCT No.: PCT/EP2014/064271

§ 371 Date: Jan. 14, 2016

(30) Foreign Application Priority Data
Jul. 19, 2013 (EP) 13177173.5

(51) Int. Cl. A61B 17/32; A61B 5/055

(52) U.S. Cl. CPC A61B 17/320068; A61B 5/055; A61B 2017/00022

(57) ABSTRACT

A high-intensity focused ultrasound therapy unit (12), in particular for ultrasound ablation treatment, comprising an ultrasound transducer probe (40) with an ultrasound transducer array (44), a guiding unit (38) that is configured for monitoring a position of the ultrasound transducer array (44) relative to a subject of interest (24), and for providing information on the monitored position to a human interface, and a control unit (42) that is configured to control at least one operational parameter of the ultrasound transducer array (44), so as to adjust the penetration depth of the focused ultrasound beam into the subject's tissue depending on a rate of change of a relative position of the ultrasound transducer array (44) relative to the subject of interest (24); and—a magnetic resonance image-guided high-intensity focused ultrasound therapy system (10), comprising a high-intensity focused ultrasound therapy unit (12) and a magnetic resonance imaging system (14) as the guiding unit (38).

### FIELD OF THE INVENTION

The invention pertains to a high-intensity focused ultrasound therapy unit, in particular for ultrasound ablation treatment, and a magnetic resonance image-guided high-intensity focused ultrasound therapy system.

### BACKGROUND OF THE INVENTION

In the field of cancer treatment, it is known to apply high-intensity focused ultrasound to malignant tumors as a method for thermal ablation of tissue. The depth of penetration of the applied ultrasound into the tissue varies with its frequency, which typically lies in the range between 0.5 MHz and 10 MHz. U.S. Pat. No. 6,488,639 B1 describes an apparatus enabling to adjust the focused ultrasound frequency according to a target attenuation, a thickness of the tissues traversed, a temperature involution, or a lesion displacement during emission. The international application WO97/22015 mentions to simply control the ultrasound transducer in that a speed limit is determined beyond which no ultrasound is generated. The European patent application EP 2 574 375 concerns a therapeutic apparatus with a HIFU system in which the sonication is controlled on the basis of a determined or predicted position of the target zone being within the deflection zone.

It is desirable to provide a therapy unit with a high degree of operating comfort for a user when carrying out a sonication of a target region.

### SUMMARY OF THE INVENTION

In one aspect of the present invention, the object is achieved by a high-intensity focused ultrasound therapy unit, in particular for ultrasound ablation treatment, comprising:
an ultrasound transducer probe having a main direction of extension and a longitudinal axis parallel to the main direction of extension, and an ultrasound transducer array arranged parallel to the longitudinal axis, wherein the ultrasound transducer probe, upon activation, is configured to apply a focused ultrasound beam of an ultrasound frequency from the ultrasound transducer array to adjacent tissue within a subject of interest;
a guiding unit that is configured for monitoring a position of the ultrasound transducer array relative to the subject of interest and for providing information on the monitored position to a human interface; and
a control unit that is configured to control at least one operational parameter of the ultrasound transducer array so as to adjust the penetration depth of the focused ultrasound beam into the subject's tissue depending on a rate of change of a relative position of the ultrasound transducer array relative to the subject of interest. According to the invention one or several operational parameters of the ultrasound transducer array are adjusted as a function of the velocity of the ultrasound transducer. Notably, the ultrasoudn frequency may be lowered as the ultrasound transducer is moved more slowly to increase the penetration depth as the ultrasound transdcuer is decelarated. Then the penetration depth gradually increases as the ultrasound transducer comes to a standstill at its final position or orientation. This achieves an accurate and comprehensive depsotion of ultrasound energy into a target zone tin the patient's tissue in a time-efficient manner. The movement may include translation, rotation or a combination of both. Accodingly, the ultrasound transducer's velocity may be its travelled linear distance per unit of time as well as its rotational velocity.

In a suitable embodiment, the high-intensity focused ultrasound therapy unit can also be employed for hypothermia purposes.

The phrase “operational parameter”, as used in this application, shall particularly encompass parameters such as level of power, frequency, or direction of propagation.

By that, an adjustment of an operational parameter of the ultrasound transducer array can be coupled to an analysis of the rate of change of the relative position of the ultrasound transducer array relative to the subject of interest. In a suitable embodiment, this can allow for options of at least partially automating a workflow of sonication of a target region within the subject of interest.

In a preferred embodiment, the ultrasound transducer probe is furnished with an acceptance member arranged in a distal portion of the ultrasound transducer probe, the acceptance member being configured for containing the ultrasound transducer array. This can be especially advantageous regarding access for a treatment of certain types of tumors, such as prostate cancer.

The phrase “distal portion”, as used in this application, shall particularly be understood as a third, more preferable as a fourth, of a length of the ultrasound transducer probe in the main direction of extension that includes a distal end of the ultrasound transducer probe.

Preferably the acceptance member is designed as a slot, or a recess, but may be designed as any other member that appears to be suitable to the person skilled in the art.

In a further embodiment, the high-intensity focused ultrasound therapy unit includes an electro-mechanical drive for rotating the ultrasound transducer probe about the longitudinal axis, and the control unit is further equipped with a first control member for enabling adjusting a rotational position of the ultrasound transducer array by a user.

By that, the user can readily transfer the ultrasound transducer array to a desired position and can check the position of the ultrasound transducer array by consulting the human interface. By example, the user may be an interventional radiologist, and the human interface may be a flat touchscreen.

The term “control member”, as used in this application, shall particularly encompass mechanical members such as knob, foot-pedal, slider, as well as a virtual soft key on a touchscreen, and also common human interface devices, such as keyboard, computer mouse, touchpad, graphics tablet, and joystick. The control member may also be designed as a dead-man's control, preferably returning to non-sonication after release, thereby providing additional safety for the subject of interest.

In another preferred embodiment, the control unit is configured to automatically adjust the at least one operational parameter if a rate of change of the position of the ultrasound transducer array relative to the subject of interest falls below a predetermined value. In this way, the power for driving the ultrasound transducer array may be lowered, and/or the frequency may be lowered to achieve a larger penetration depth if the relative position of the ultrasound transducer array is stationary. The predetermined value may reside in a memory unit which the control unit has access to.

A larger penetration depth of the applied ultrasound in a situation in which the relative position of the ultrasound transducer array is stationary can readily be accomplished if the adjustment of the at least one operational parameter is proportional to a time duration during which the rate of change of the relative position of the ultrasound transducer array relative to the subject of interest has fallen below the predetermined value.

In yet another preferred embodiment, the control unit comprises a second control member for enabling adjusting the ultrasound frequency by a user, wherein the control unit is configured to automatically reduce a total power to be emitted by the ultrasound transducer array with increasing ultrasound frequency. In this way, a constant volumetric density of heat applied to the tissue is achievable, resulting in a constant heating despite the adjustment of the ultrasound frequency by the user.

In still another embodiment, the control unit comprises a third control member for enabling adjusting the total power emitted by the ultrasound transducer array by the user. In this way, the user can have direct control over a heating generated in the tissue by the ultrasound transducer array.

In another preferred embodiment, the control unit comprises a power divider unit and is configured to divide a selected power among individual ultrasound transducers of the ultrasound transducer array via the power divider unit for modifying a shape of the focused ultrasound beam generated by the ultrasound transducer array. The control unit comprises a fourth control member for enabling adjusting the shape of the focused ultrasound beam generated by the ultrasound transducer array by a user. By that, the user is provided full control over the shape of the focused ultrasound beam and a heating zone generated by it, and is enabled to individually adapt the beam shape and the heating zone to a target region of the subject of interest. Modifying the shape of the focused ultrasound beam shall also encompass a division of the selected power among the individual ultrasound transducers in such a manner that individual ultrasound transducer selected by the user may completely be switched off. The user may control the shape of the beam either before activation of the ultrasound sensors array or during sonication.

In yet another embodiment, the control unit comprises a fifth control member provided to a user for enabling selecting ultrasound transducers of the ultrasound transducer array to be activated. In this way, the user is enabled to vary a length in the direction of the longitudinal axis of ultrasound transducers to be activated for adaption to a size of a target region within the subject of interest. This is particularly advantageous for ablating a limited section in a controlled way. This condition may, by way of example, occur in a typical ablation treatment of prostate tissue.

In another aspect of the invention, a magnetic resonance image-guided high-intensity focused ultrasound therapy system is provided, comprising a magnetic resonance imaging system that is provided for acquiring magnetic resonance imaging data from at least a portion of a subject of interest, and a high-intensity focused ultrasound therapy unit pursuant to any one of the embodiments disclosed herein, or a combination thereof.

The magnetic resonance imaging system comprises a magnetic resonance scanner, an examination space provided to position a subject of interest within, a main magnet provided for generating a static magnetic field at least in the examination space, a magnetic gradient coil system provided for generating gradient magnetic fields superimposed to the static magnetic field, an image processing unit configured to image the portion of the subject of interest by processing the acquired magnetic resonance imaging data of the portion of the subject of interest, and a magnetic resonance imaging control unit that is provided for controlling functions of the magnetic resonance imaging system. The magnetic resonance imaging system functions as the guiding unit of the high-intensity focused ultrasound therapy system.

If the magnetic resonance imaging system is configured to provide thermographic magnetic resonance image information of a magnetic resonance imaging plane that coincides with a plane in which the applied focused ultrasound beam from the ultrasound sensor array is lying, precise and direct information on a temperature development in the target region of the subject of interest during treatment can be provided to the user.

Preferably, the thermographic magnetic resonance image information is derived by proton resonance frequency shift thermometry. Methods for carrying out proton resonance frequency shift thermometry are well known to the person skilled in the art. The image processing unit may have a software module that is implementable in a memory unit and executable in a processing unit of the image processing unit, the software module comprising steps in the form of a program code for carrying out proton resonance frequency shift thermometry from acquired magnetic resonance imaging data of the portion of the subject of interest.

### BRIEF DESCRIPTION OF THE DRAWINGS

These and other aspects of the invention will be apparent from and elucidated with reference to the embodiments described hereinafter. Such embodiment does not necessarily represent the full scope of the invention, however, and reference is made therefore to the claims and herein for interpreting the scope of the invention.

In the drawings:

FIG. 1 is a schematic illustration of a part of an embodiment of a magnetic resonance image-guided high-intensity focused ultrasound therapy system in accordance with the invention, and

FIG. 2 illustrates details of the high-intensity focused ultrasound therapy unit of the ultrasound therapy system pursuant to FIG. 1 in a schematic way.

### DETAILED DESCRIPTION OF EMBODIMENTS

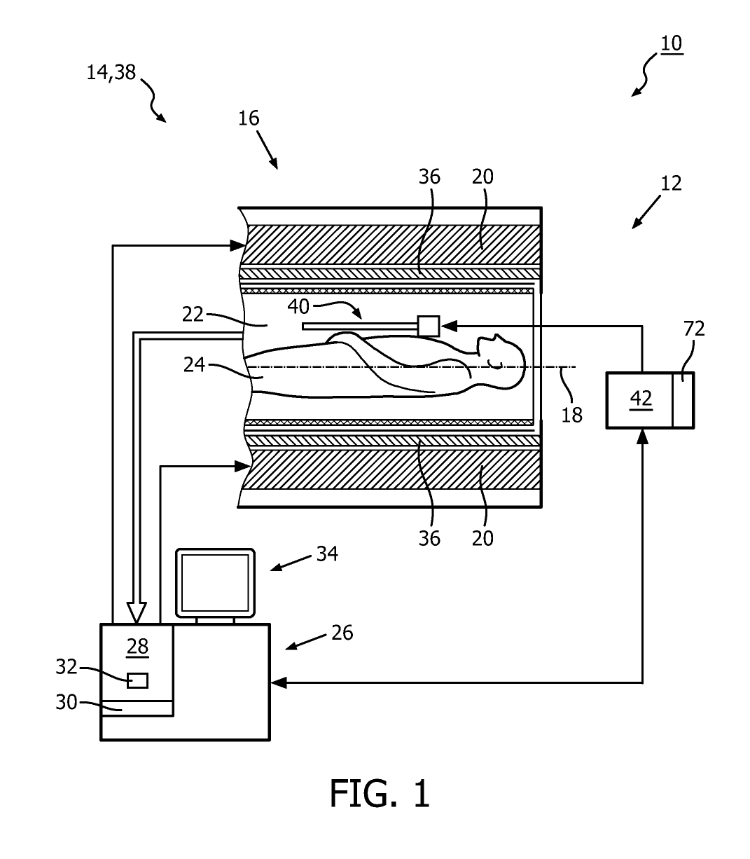

FIG. 1 shows a schematic partial illustration of a magnetic resonance (MR) image-guided high-intensity focused ultrasound (HIFU) therapy system 10 in accordance with the invention.

The magnetic resonance image-guided high-intensity focused ultrasound (MR HIFU) therapy system 10 comprises a magnetic resonance imaging system 14 provided for acquiring magnetic resonance imaging data from at least a portion of a subject of interest 24, usually a patient. The magnetic resonance imaging system 14 includes a magnetic resonance scanner 16 comprising a main magnet 20 with a center bore that defines an examination space 22 provided to position the subject of interest 24 within. A patient table has been omitted in FIG. 1 for reasons of clarity. The main magnet 20 is provided for generating a static magnetic field at least in the examination space 22, wherein the substantially static magnetic field is directed substantially parallel to a center axis 18 of the examination space 22.

Further, the magnetic resonance imaging system 14 comprises a magnetic gradient coil system 36 for generating gradient magnetic fields superimposed to the static magnetic field. The magnetic gradient coil system 36 is concentrically arranged within the bore of the main magnet 20, as is well known in the art.

The magnetic resonance imaging system 14 further includes a magnetic resonance imaging system control unit 26 with a monitoring unit 34, to control functions of the magnetic resonance scanner 16, as is commonly known in the art, and an image processing unit 28 configured to image the portion of the subject of interest 24 by processing the acquired magnetic resonance imaging data of the portion of the subject of interest 24.

The magnetic resonance imaging system 14 is configured to provide thermographic magnetic resonance image information of a selected magnetic resonance imaging plane. To this end, the image processing unit 28 includes a memory unit 30 and a processing unit 32, wherein a software module that comprises steps in the form of a program code for carrying out proton resonance frequency shift thermometry from acquired magnetic resonance imaging data of the portion of the subject of interest 24, is residing in the memory unit 30 and is executable by the processing unit 32. The thermographic magnetic resonance image information of the selected magnetic resonance imaging plane is provided to a user by repeatedly displaying updated thermographic information on the monitoring unit 34.

The MR image-guided HIFU therapy system 10 further comprises a HIFU therapy unit 12 provided for ultrasound ablation treatment in a target zone within the subject of interest 24. The target zone is defined as the tissue volume within the subject of interest 24 in which 95% of the energy would be deposited if the HIFU therapy unit 12 was activated.

The person skilled in the art may note that the HIFU therapy unit 12 may also be utilized as a hyperthermia device, for depositing energy in the target zone in order to activate tissue for improved response to e.g. chemotherapy.

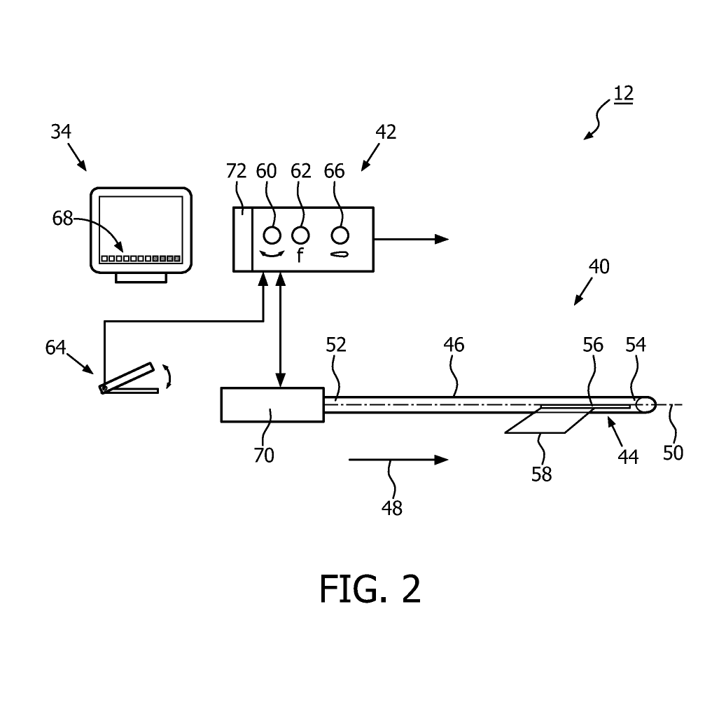

In the embodiment of FIG. 1, the HIFU therapy unit 12 comprises an ultrasound transducer probe 40, and a control unit 42. In the illustration of FIG. 1, the ultrasound transducer probe 40 is not shown in its final operating position. FIG. 2 schematically shows respective details of the HIFU therapy unit 12.

The ultrasound transducer probe 40 has a rod 46 with a main direction of extension 48, a longitudinal axis 50 parallel to the main direction of extension 48, an end 52 that is proximal to the user, and another end 54 that is distal to the user. The ultrasound transducer probe 40 includes a plurality of ultrasound transducers arranged in an array 44 parallel to the longitudinal axis 50. Further, the rod 46 is furnished with an acceptance member 56 arranged in a portion of the rod 46 that includes the distal end 54. The acceptance member 56 is formed as a slot, which in an operational state is configured for containing the ultrasound transducer array 44. The ultrasound transducer probe 40, upon activation, is configured to apply a focused ultrasound beam of an ultrasound frequency, from the ultrasound transducer array 44 to adjacent tissue within the subject of interest.

The ultrasound transducer probe 40 includes an electro-mechanical drive 70 for rotating the ultrasound transducer probe 40 about the longitudinal axis 50. To this end, the rod 46 is rotatably supported by the electro-mechanical drive 70. The control unit 42 is equipped with a first control member 60 designed as a rotary knob for enabling adjusting a rotational position of the ultrasound transducer array 44 by the user in a first operational mode.

The control unit 42 is further configured to provide electric power to the ultrasound transducer array 44 and to control at least one operational parameter of the ultrasound transducer array 44 depending on a rate of change of a relative position of the ultrasound transducer array 44 relative to the subject of interest 24, as will be described in more detail in the following. In order to facilitate the control of the operational parameters, the control unit 42 has several operational modes. It shall be understood that the operational modes are meant to be arbitrarily selectable as options.

If, in the first operational mode of the control unit 42, the user keeps the rotational position of the ultrasound transducer array 44 stationary, so that a rate of change of the position of the ultrasound transducer array 44 relative to the subject of interest 24 falls below a predetermined value that is stored in a memory unit of the control unit 42, the control unit 42 is configured to automatically adjust an operational parameter given by the ultrasound frequency, and, more specific, to lower it in order to achieve a deeper penetration of the ultrasound beam in the tissue of the subject of interest 24. The adjustment of the ultrasound frequency is proportional to a time duration during which the rate of change of the relative position of the ultrasound transducer array 44 relative to the subject of interest 24 has fallen below the predetermined value.

The control unit 42 comprises a second control member 62 that can be used in a second operational mode. The second control member 62 is designed as another rotary knob and enables adjusting the ultrasound frequency by the user for changing the penetration depth of the ultrasound beam, as is obvious to the person skilled in the art. In the second operational mode, the control unit 42 is configured to automatically reduce a total power emitted by the ultrasound transducer array 44 with increasing ultrasound frequency. The amount of reduction for a specific adjustment of the ultrasound frequency is determined based on a lookup table that is stored in the memory unit of the control unit 42.

In a third operational mode of the control unit 42, a third control member 64 of the control unit 42 is activated for enabling the user to adjust the total power to be emitted by the ultrasound transducer array 44, so as to control a heating rate of the tissue adjacent to the ultrasound transducer array 44. The third control member 64 is formed by a foot-pedal with a dead-man's control that automatically returns to non-sonication if released by the user.

In a fourth operational mode of the control unit 42, a fourth control member 66 of the control unit 42 is provided by another rotary knob for enabling the user to adjust the shape of the focused ultrasound beam generated by the ultrasound transducer array 44. To this end, the control unit 42 comprises a power divider unit 72 and is configured to divide a selected power among individual ultrasound transducers of the ultrasound transducer array 44 via the power divider unit 72 so as to shape the focused ultrasound beam generated by the ultrasound transducer array 44. By way of example, the rotary knob may have two end positions. In the one of the two end positions, the power divider unit 72 may evenly distribute the power among all ultrasound transducers of the ultrasound transducer array 44, which results in a broad beam. In the other of the two end positions, the power divider unit 72 may transmit the power in such a way that a portion of the power transmitted to an individual ultrasound transducer is the lower the larger a distance of the individual ultrasound transducer is from a center position of the ultrasound transducer array 44, resulting in a narrow beam.

In a fifth operational mode of the control unit 42, a fifth control member 68 of the control unit 42 is provided by a plurality of toggle soft keys which are aligned in a lower part of the touch screen of the monitoring unit 34, wherein each toggle soft key represents a number of ultrasound transducers of the ultrasound transducer array 44. By touching one of the toggle soft keys, the user can switch the respective ultrasound transducers on or off, so that the user can intuitively select ultrasound transducers that are to be activated by touching the soft keys on the touch screen. In this way, an active length of the ultrasound transducer array 44 can be shortened for a treatment of small portions of tissue within the subject of interest 24.

During treatment, the ultrasound beam generated by the ultrasound transducer array 44 defines a plane of ultrasound emission 58, adjacent to which the tissue of the subject of interest 24 is heated up. The magnetic resonance imaging system 14 is configured to provide thermographic magnetic resonance image information in a plane that coincides with the plane of ultrasound emission 58, and provides information on a temperature evolution in this plane by displaying a temperature map overlying a magnetic resonance image of the same plane on the touch screen of the monitoring unit 34. In this way, the magnetic resonance imaging system 14 is provided as a guiding unit 38 of the HIFU therapy unit 12, wherein the magnetic resonance imaging system 14 is configured for monitoring a position of the ultrasound transducer array 44 relative to the subject of interest 24 and for providing information on the monitored position to a human interface and on the temperature evolution during treatment.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

REFERENCE SYMBOL LIST

| | |
|---|---|
| 10 | MR HIFU therapy system |
| 12 | HIFU therapy unit |
| 14 | magnetic resonance imaging system |
| 16 | magnetic resonance scanner |
| 18 | center axis |
| 20 | main magnet |
| 22 | examination space |
| 24 | subject of interest |
| 26 | magnetic resonance imaging system control unit |
| 28 | image processing unit |
| 30 | memory unit |
| 32 | processing unit |
| 34 | monitoring unit |
| 36 | magnetic gradient coil system |
| 38 | guiding unit |
| 40 | ultrasound transducer probe |
| 42 | control unit |
| 44 | ultrasound transducer array |
| 46 | rod |
| 48 | main direction of extension |
| 50 | longitudinal axis |
| 52 | proximal end |
| 54 | distal end |
| 56 | acceptance member |
| 58 | plane of ultrasound emission |
| 60 | first control member |
| 62 | second control member |
| 64 | third control member |
| 66 | fourth control member |
| 68 | fifth control member |
| 70 | electro-mechanical drive |
| 72 | power divider unit |

1. A high-intensity focused ultrasound therapy unit, for ultrasound ablation treatment, comprising:
an ultrasound transducer probe having a main direction of extension and a longitudinal axis parallel to the main direction of extension, and an ultrasound transducer array arranged parallel to the longitudinal axis, wherein the ultrasound transducer probe, upon activation, is configured to apply a focused ultrasound beam of an ultrasound frequency from the ultrasound transducer array to adjacent tissue within a subject of interest;
a guiding unit that is configured for monitoring a position of the ultrasound transducer array relative to the subject of interest and for providing information on the monitored position to a human interface; and
a control unit that is configured to control at least one operational parameter of the ultrasound transducer array, so as to adjust the penetration depth of the focused ultrasound beam into the subject's tissue depending on a rate of change of a relative position of the ultrasound transducer array relative to the subject of interest.

2. The high-intensity focused ultrasound therapy unit of claim 1, wherein the ultrasound transducer probe is furnished with an acceptance member arranged in a distal portion of the ultrasound transducer probe, the acceptance member being configured for containing the ultrasound transducer array.

3. The high-intensity focused ultrasound therapy unit of claim 1, further including an electro-mechanical drive for rotating the ultrasound transducer probe about the longitudinal axis, wherein the control unit is further equipped with a first control member for enabling adjusting a rotational position of the ultrasound transducer array by a user.

4. The high-intensity focused ultrasound therapy unit of claim 1, wherein the control unit is configured to automatically adjust the at least one operational parameter if a rate of change of the position of the ultrasound transducer array relative to the subject of interest falls below a predetermined value.

5. The high-intensity focused ultrasound therapy unit of claim 4, wherein adjustment of the at least one operational parameter is proportional to a time duration during which the rate of change of the relative position of the ultrasound transducer array relative to the subject of interest has fallen below the predetermined value.

6. The high-intensity focused ultrasound therapy unit of claim 1, wherein the control unit comprises a second control member for enabling adjusting the ultrasound frequency by a user, wherein the control unit is configured to automatically reduce a total power to be emitted by the ultrasound transducer array with increasing ultrasound frequency.

7. The high-intensity focused ultrasound therapy unit of claim 1, wherein the control unit comprises a third control member for enabling adjusting the total power emitted by the ultrasound transducer array by a user.

8. The high-intensity focused ultrasound therapy unit of claim 1, wherein the control unit comprises a power divider unit and is configured to divide a selected power among individual ultrasound transducers of the ultrasound transducer array via the power divider unit for modifying a shape of the focused ultrasound beam generated by the ultrasound transducer array, and wherein the control unit comprises a fourth control member for enabling adjusting the shape of the focused ultrasound beam generated by the ultrasound transducer array by a user.

9. The high-intensity focused ultrasound therapy unit of claim 1, wherein the control unit comprises a fifth control member provided to a user for enabling selecting ultrasound transducers of the ultrasound transducer array to be activated.

10. A magnetic resonance image-guided high-intensity focused ultrasound therapy system, comprising:
a magnetic resonance imaging system provided for acquiring magnetic resonance imaging data from at least a portion of a subject of interest, comprising:
a magnetic resonance scanner;
an examination space provided to position the subject of interest within;
a main magnet provided for generating a static magnetic field at least in the examination space;
a magnetic gradient coil system provided for generating gradient magnetic fields superimposed to the static magnetic field;
an image processing unit configured to image the portion of the subject of interest by processing the acquired magnetic resonance imaging data of the portion of the subject of interest;
a magnetic resonance imaging control unit that is provided for controlling functions of the magnetic resonance imaging system; and
the high-intensity focused ultrasound therapy unit as claimed in claim 1, wherein the magnetic resonance imaging system is configured as the guiding unit.

11. The magnetic resonance image-guided high-intensity focused ultrasound therapy system of claim 10, wherein the magnetic resonance imaging system is configured to provide thermographic magnetic resonance image information of a magnetic resonance imaging plane that coincides with a plane in which the applied focused ultrasound beam from the ultrasound sensor array is lying.

12. The magnetic resonance image-guided high-intensity focused ultrasound therapy system of claim 10, wherein the thermographic magnetic resonance image information is derived by proton resonance frequency shift thermometry.

13. A magnetic resonance image-guided high-intensity focused ultrasound therapy system comprising:
a high-intensity focused ultrasound therapy unit comprising an ultrasound transducer probe having a main direction of extension and a longitudinal axis parallel to the main direction of extension, and an ultrasound transducer array arranged parallel to the longitudinal axis, wherein the ultrasound transducer probe, upon activation, is configured to apply a focused ultrasound beam of an ultrasound frequency from the ultrasound transducer array to adjacent tissue within a subject of interest;
a control unit configured to control at least one operational parameter of the ultrasound transducer array, so as to adjust the penetration depth of the focused ultrasound beam into the subject's tissue depending on a rate of change of a relative position of the ultrasound transducer array relative to the subject of interest; and
a magnetic resonance imaging system configured to monitor a position of the ultrasound transducer array relative to the subject of interest and for providing information on the monitored position to a human interface.

14. A high-intensity focused ultrasound therapy unit comprising:
an ultrasound transducer probe having a main direction of extension and a longitudinal axis parallel to the main direction of extension, and an ultrasound transducer array arranged parallel to the longitudinal axis, wherein the ultrasound transducer probe, upon activation, is configured to apply a focused ultrasound beam of an ultrasound frequency from the ultrasound transducer array to adjacent tissue within a subject of interest; and
a control unit that is configured to control at least one operational parameter of the ultrasound transducer array, so as to adjust the penetration depth of the focused ultrasound beam into the subject's tissue depending on a rate of change of a relative position of the ultrasound transducer array relative to the subject of interest.

15. The high-intensity focused ultrasound therapy unit of claim 14, further including a guiding unit that is configured for monitoring a position of the ultrasound transducer array relative to the subject of interest and for providing information on the monitored position to a human interface.

16. The high-intensity focused ultrasound therapy unit of claim 15, wherein the guiding unit is a magnetic resonance imaging system.

17. The high-intensity focused ultrasound therapy unit of claim 15, wherein the ultrasound transducer probe is furnished with an acceptance member arranged in a distal portion of the ultrasound transducer probe, the acceptance member configured to contain the ultrasound transducer array.

18. The high-intensity focused ultrasound therapy unit of claim 15, wherein the control unit is configured to automatically adjust the at least one operational parameter if a rate of change of the position of the ultrasound transducer array relative to the subject of interest falls below a predetermined value.

19. The high-intensity focused ultrasound therapy unit of claim 15, wherein the control unit comprises a second control member for enabling adjusting the ultrasound frequency by a user, wherein the control unit is configured to automatically reduce a total power to be emitted by the ultrasound transducer array with increasing ultrasound frequency or a third control member for enabling adjusting the total power emitted by the ultrasound transducer array by a user.

20. The high-intensity focused ultrasound therapy unit of claim 15, wherein the control unit comprises a power divider unit and is configured to divide a selected power among individual ultrasound transducers of the ultrasound transducer array via the power divider unit for modifying a shape of the focused ultrasound beam generated by the ultrasound transducer array, and wherein the control unit comprises a fourth control member for enabling adjusting the shape of the focused ultrasound beam generated by the ultrasound transducer array by a user.
